(19) United States
(12) Patent Application Publication
(10) Pub. No.: US 2026/0258419 A1
(43) Pub. Date: Sep. 3, 2026

(54) RECOMBINANT ADENO-ASSOCIATED VIRUS DELIVERY OF EXON 2-TARGETED U7SNRNA POLYNUCLEOTIDE CONSTRUCTS

(71) Applicant: RESEARCH INSTITUTE AT NATIONWIDE CHILDREN'S HOSPITAL, Columbus, OH (US)

(72) Inventors: Kevin Flanigan, Columbus, OH (US); Adeline Vulin-Chaffiol, Columbus, OH (US); Nicolas Wein, Columbus, OH (US)

(21) Appl. No.: 19/678,374

(22) Filed: May 15, 2026

Related U.S. Application Data
(62) Division of application No. 17/546,428, filed on Dec. 9, 2021, now Pat. No. 12,655,427.
Application No. 17/546,428 is a division of application No. 16/032,744, filed on Jul. 11, 2018, now Pat. No. 11,230,707.
Application No. 16/032,744 is a continuation of application No. 15/825,841, filed on Nov. 29, 2017, now abandoned.
Application No. 15/825,841 is a continuation of application No. 14/785,769, filed on Apr. 18, 2014, now Pat. No. 9,862,945.
(60) Provisional application No. 61/814,256, filed on Apr. 20, 2013.

(51) Int. Cl. C12N 15/113; A61K 48/00; C07K 14/47; C12N 7/00; C12N 15/11; C12N 15/86; C12N 15/864

(52) U.S. Cl. CPC C12N 15/113; A61K 48/005; C07K 14/4708; C12N 7/00; C12N 15/111; C12N 15/86; C12N 15/8645; C12N 2310/11; C12N 2320/32; C12N 2320/33; C12N 2330/51; C12N 2750/14132; C12N 2750/14143; C12N 2750/14171; C12N 2750/14321; C12N 2750/14333

(57) ABSTRACT

The present invention relates to recombinant adeno-associated virus (rAAV) delivery of polynucleotides for treating Duchenne Muscular Dystrophy resulting from the duplication of DMD exon 2. The invention provides rAAV products and methods of using rAAV in the treatment of Duchenne Muscular Dystrophy.

This application is a division of U.S. patent application Ser. No. 17/546,428 filed Dec. 9, 2021, which is a division of U.S. patent application Ser. No. 16/032,744 filed Jul. 11, 2018 (issued U.S. Pat. No. 11,230,707), which is a continuation of U.S. patent application Ser. No. 15/825,841 filed Nov. 29, 2017, which is a continuation of U.S. patent application Ser. No. 14/785,769 filed Oct. 20, 2015 (issued U.S. Pat. No. 9,662,945), which is a national stage entry of International Patent Application No. PCT/US2014/034702 filed Apr. 18, 2014, which claims the benefit of U.S. Provisional Patent Application No. 61/814,256 filed Apr. 19, 2013, all of which are incorporated by reference herein in their entireties.

### FIELD OF THE INVENTION

The present invention relates to recombinant adeno-associated virus (rAAV) delivery of polynucleotides for treating Duchenne Muscular Dystrophy resulting from the duplication of DMD exon 2. The invention provides rAAV products and methods of using the rAAV in the treatment of Duchenne Muscular Dystrophy.

### INCORPORATION BY REFERENCE OF THE SEQUENCE LISTING

This application contains, as a separate part of disclosure, a Sequence Listing in computer-readable form (Filename: 47699D_SeqListing.xml; Size: 17,021 bytes; Created: May 14, 2026), which is incorporated by reference herein in its entirety.

### BACKGROUND

Muscular dystrophies (MDs) are a group of genetic diseases. The group is characterized by progressive weakness and degeneration of the skeletal muscles that control movement Some forms of MD develop in infancy or childhood, while others may not appear until middle age or later. The disorders differ in terms of the distribution and extent of muscle weakness (some forms of MD also affect cardiac muscle), the age of onset, the rate of progression, and the pattern of inheritance.

One form of MD is Duchenne Muscular Dystrophy (DMD). It is the most common severe childhood form of muscular dystrophy affecting 1 in 5000 newborn males. DMD is caused by mutations in the DMD gene leading to absence of dystrophin protein (427 KDa) in skeletal and cardiac muscles, as well as GI tract and retina. Dystrophin not only protects the sarcolemma from eccentric contractions, but also anchors a number of signaling proteins in close proximity to sarcolemma. Many clinical cases of DMD are linked to deletion mutations in the DMD gene. Despite many lines of research following the identification of the DMD gene, treatment options are limited. Corticosteroids are clearly beneficial but even with added years of ambulation the benefits are offset by long-term side effects. The original controlled, randomized, double-blind study reported more than 20 years ago showed benefits using prednisone [Mendell et al., N. Engl. J. Med., 320: 1592-1597 (1989)]. Subsequent reports showed equal efficacy using deflazacort, a sodium-sparing steroid [Biggar et al., J. Pediatr., 138: 45-50 (2001)]. Recent studies also demonstrate efficacy by exon skipping, prolonging walking distance on the 6MWT. Thus far, published clinical studies have reported benefit for only mutations where the reading frame is restored by skipping exon 51 [Cirak et al., Lancet, 378: 595-605 (2011) and Goemans et al., New Engl. J. Med. 364: 1513-1522 (2011)]. In the only report of a double blind, randomized treatment trial promising results were demonstrated with eteplirsen, a phosphorodiamidate morpholino oligomer (PMO). In all of these exon-skipping trials, the common denominator of findings has been a plateau in walking ability after an initial modest improvement.

See also, U.S. Patent Application Publication Nos. 2012/0077860 published Mar. 29, 2012; 2013/0072541 published Mar. 21, 2013; and 2013/0045538 published Feb. 21, 2013.

In contrast to the deletion mutations, DMD exon duplications account for around 5% of disease-causing mutations in unbiased samples of dystrophinopathy patients [Dent et al., Am. J. Med. Genet., 134(3): 295-298 (2005)], although in some catalogues of mutations the number of duplications is higher [including that published by the United Dystrophinopathy Project in Flanigan et al., Hum. Mutat., 30(12): 1657-1666 (2009), in which it was 11%].

Adeno-associated virus (AAV) is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 {1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

An AAV8-like AAV termed rh.74 to deliver DNAs encoding various proteins. Xu et al., Neuromuscular Disorders, 17: 209-220 (2007) and Martin et al., Am. J. Physiol. Cell. Physiol., 296: 476-488 (2009) relate to rh.74 expression of cytotoxic T cell GalNAc transferase for Duchenne muscular dystrophy. Rodino-Klapac et al., Mol. Ther., 18(1): 109-117 (2010) describes AAV rh.74 expression of a micro-dystrophin FLAG protein tag fusion after delivery of the AAV rh.74 by vascular limb perfusion.

The muscular dystrophies are a group of diseases without identifiable treatment that gravely impact individuals, families, and communities. The costs are incalculable. Individuals suffer emotional strain and reduced quality of life associated with loss of self-esteem. Extreme physical challenges resulting from loss of limb function creates hardships in activities of daily living. Family dynamics suffer through financial loss and challenges to interpersonal relationships. Siblings of the affected feel estranged, and strife between spouses often leads to divorce, especially if responsibility for the muscular dystrophy can be laid at the feet of one of the parental partners. The burden of quest to find a cure often becomes a life-long, highly focused effort that detracts and challenges every aspect of life. Beyond the family, the community bears a financial burden through the need for added facilities to accommodate the handicaps of the muscular dystrophy population in special education, special transportation, and costs for recurrent hospitalizations to treat recurrent respiratory tract infections and cardiac complications. Financial responsibilities are shared by state and federal governmental agencies extending the responsibilities to the taxpaying community.

There thus remains a need in the art for treatments for muscular dystrophies including DMD.

### DESCRIPTION

The present invention provides methods and products for preventing, delaying the progression of, and/or treating DMD involving a duplication of exon 2 of the DMD gene. The methods involve using AAV as a delivery vector for a polynucleotide construct encoding a U7 small nuclear RNA and an exon 2 targeting antisense sequence, an “exon 2-targeted U7snRNA polynucleotide construct.” For example, the polynucleotide construct is inserted in the genome of a rAAV rh.74, the genome of a rAAV6 or the genome of a rAAV9. The polynucleotide sequence of the AAV rh.74 genome is shown in FIGS. 7A-7K and SEQ ID NO: 1.

Exemplary exon 2 targeting antisense sequences include, but are not limited to,

U7B
(SEQ ID NO: 3)
TCAAAAGAAAACATTCACAAAATGGGTA;
U7Along
(SEQ ID NO: 4)
GTTTTCTTTTGAAGATCTTCTCTTTCATcta;
U7Ashort
(SEQ ID NO: 5)
AGATCTTCTCTTTCATcta;
and
U7C
(SEQ ID NO: 6)
GCACAATTTTCTAAGGTAAGAAT.

In one aspect, a method of ameliorating DMD in a patient is provided. In some embodiments, the method comprises the step of administering a rAAV to the patient, wherein the genome of the rAAV comprises an exon 2-targeted U7snRNA polynucleotide construct.

In yet another aspect, the invention provides a method of inhibiting the progression of dystrophic pathology associated with DMD. In some embodiments, the method comprises the step of administering a rAAV to the patient, wherein the genome of the rAAV comprises an exon 2-targeted U7snRNA polynucleotide construct.

In still another aspect, a method of improving muscle function in a patient afflicted with DMD is provided. In some embodiments, the method comprises the step of administering a rAAV to the patient, wherein the genome of the rAAV comprises an exon 2-targeted U7snRNA polynucleotide construct. In some instances, the improvement in muscle function is an improvement in muscle strength. The improvement in muscle strength is determined by techniques known in the art such as the maximal voluntary isometric contraction testing (MVICT). In some instances, the improvement in muscle function is an improvement in stability in standing and walking. The improvement in stability strength is determined by techniques known in the art such as the 6-minute walk test (6MWT) or timed stair climb.

In another aspect, the invention provides a method of delivering an exon 2-targeted U7snRNA polynucleotide construct to an animal (including, but not limited to, a human). In some embodiments, the method comprises the step of a rAAV to the patient, wherein the genome of the rAAV comprises an exon 2-targeted U7snRNA polynucleotide construct.

Cell transduction efficiencies of the methods of the invention described above and below may be at least about 60, 65, 70, 75, 80, 85, 90 or 95 percent.

In some embodiments of the foregoing methods of the invention, the virus genome is a self-complementary genome. In some embodiments of the methods, the genome of the rAAV lacks AAV rep and cap DNA. In some embodiments of the methods, the rAAV is a SC rAAV U7_ACCA comprising the exemplary genome set out in FIG. 9. In some embodiments the rAAV is a rAAV rh.74. In some embodiments, the rAAV is a rAAV6. In some embodiments, the rAAV is a rAAV9.

In yet another aspect, the invention provides a rAAV comprising the AAV rh.74 capsid and a genome comprising the exemplary exon 2-targeted U7 snRNA polynucleotide construct U7_ACCA. In some embodiments, the genome of the rAAV lacks AAV rep and cap DNA. In some embodiments, the rAAV comprises a self-complementary genome. In some embodiments of the methods, the rAAV is a SC rAAV U7_ACCA comprising the exemplary genome is set out in FIG. 9. In some embodiments the rAAV is a rAAV rh.74. In some embodiments, the rAAV is a rAAV6. In some embodiments, the rAAV is a rAAV9.

Recombinant AAV genomes of the invention comprise one or more AAV ITRs flanking at least one exon 2-targeted U7 snRNA polynucleotide construct. Genomes with exon 2-targeted U7 snRNA polynucleotide constructs comprising each of the exon 2 targeting antisense sequences are specifically contemplated, as well as genomes with exon 2-targeted U7 snRNA polynucleotide constructs comprising each possible combination of two or more of the exon 2 targeting antisense sequences. In some embodiments, including the exemplified embodiments, the U7 snRNA polynucleotide includes its own promoter. AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10 and AAV-11. As noted in the Background section above, the nucleotide sequences of the genomes of various AAV serotypes are known in the art. In some embodiments of the invention, the promoter DNAs are muscle-specific control elements, including, but not limited to, those derived from the actin and myosin gene families, such as from the myoD gene family [See Weintraub et al., Science, 251: 761-766 (1991)], the myocyte-specific enhancer binding factor MEF-2 [Cserjesi and Olson, Mol. Cell. Biol., 11: 4854-4862 (1991)], control elements derived from the human skeletal actin gene [Muscat et al., Mol. Cell. Biol., 7: 4089-4099 (1987)], the cardiac actin gene, muscle creatine kinase sequence elements [Johnson et al., Mol. Cell. Biol., 9:3393-3399 (1989)] and the murine creatine kinase enhancer (MCK) element, desmin promoter, control elements derived from the skeletal fast-twitch troponin C gene, the slow-twitch cardiac troponin C gene and the slow-twitch troponin I gene: hypozia-inducible nuclear factors [Semenza et al., Proc. Natl. Acad. Sci. USA, 88: 5680-5684 (1991)], steroid-inducible elements and promoters including the glucocorticoid response element (GRE) [See Mader and White, Proc. Natl. Acad. Sci. USA, 90: 5603-5607 (1993)], and other control elements.

DNA plasmids of the invention comprise rAAV genomes of the invention. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10 and AAV-11. Use of cognate components is specifically contemplated. Production of pseudotyped rAAV is disclosed in, for example, WO 01/83692 which is incorporated by reference herein in its entirety.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. The foregoing documents are hereby incorporated by reference in their entirety herein, with particular emphasis on those sections of the documents relating to rAAV production.

The invention thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

In another embodiment, the invention contemplates compositions comprising rAAV of the present invention. Compositions of the invention comprise rAAV in a pharmaceutically acceptable carrier. The compositions may also comprise other ingredients such as diluents. Acceptable carriers and diluents are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Titers of rAAV to be administered in methods of the invention will vary depending, for example, on the particular rAAV, the mode of administration, the treatment goal, the individual, and the cell type(s) being targeted, and may be determined by methods standard in the art Titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg) (i.e., 1×10⁷ vg, 1×10⁸ vg, 1×10⁹ vg 1×10¹⁰ vg, 1×10¹¹ vg, 1×10¹² vg, 1×10¹³ vg, 1×10¹⁴ vg, respectively).

Methods of transducing a target cell (e.g., a skeletal muscle) with rAAV, in vivo or in vitro, are contemplated by the invention. The methods comprise the step of administering an effective dose, or effective multiple doses, of a composition comprising a rAAV of the invention to an animal (including a human being) in need thereof. If the dose is administered prior to development of DMD, the administration is prophylactic. If the dose is administered after the development of DMD, the administration is therapeutic. In embodiments of the invention, an effective dose is a dose that alleviates (eliminates or reduces) at least one symptom associated with DMD being treated, that slows or prevents progression to DMD, that slows or prevents progression of a disorder/disease state, that diminishes the extent of disease, that results in remission (partial or total) of disease, and/or that prolongs survival.

Administration of an effective dose of the compositions may be by routes standard in the art including, but not limited to, intramuscular, parenteral, intravenous, oral, buccal, nasal, pulmonary, intracranial, intraosseous, intraocular, rectal, or vaginal. Route(s) of administration and serotype(s) of AAV components of rAAV (in particular, the AAV ITRs and capsid protein) of the invention may be chosen and/or matched by those skilled in the art taking into account the infection and/or disease state being treated and the target cells/tissue(s). In some embodiments, the route of administration is intramuscular. In some embodiments, the route of administration is intravenous.

Combination therapies are also contemplated by the invention. Combination as used herein includes simultaneous treatment or sequential treatments. Combinations of methods of the invention with standard medical treatments (e.g., corticosteroids and/or immunosuppressive drugs) are specifically contemplated, as are combinations with other therapies such as those mentioned in the Background section above.

### BRIEF DESCRIPTION OF THE DRAWING

FIGS. 1A-1F show histology and immunofluorescence analysis of muscles in the Dup2 mouse.

FIGS. 2A-2C show immunoblots from Western blot analysis of muscles in the Dup 2 mouse.

FIG. 3 shows that skipping of a duplicated exon 2 in a MyoD-transdifferentiated myoblast induced by an AON directed at an exon splice enhancer results in 39% wild type transcript. Dosage per lane shown in nMoles (25, 50, 100, 200, 300, 400, 500). The amount of the varying transcripts are shown under each lane, with the maximum shaded. TB=transfection buffer. NSM=normal skeletal muscle. The percentage of exon 2 duplication, wt, and exon 2 deletion is listed below each lane.

FIG. 4 illustrates the U7snRNA vector approach to exon skipping. U7snRNA is used as a carrier to target the pre-messenger RNA. It is composed of a loop used for the nucleocytoplasmic export, a recognition sequence to bind the Sm proteins used for an efficient assembly between the U7snRNA and the target pre-mRNA and an antisense sequence to target the pre-mRNA. It has its own promoter and 3′ downstream sequences. The U7 cassette is then cloned in an AAV plasmid, to produce the vector.

FIG. 5 shows RT-PCR results for exon-skipping experiments using SC rAAV vectors to transduce Dup2 immortalized human fibromyoblasts with exemplary exon 2-targeted U7snRNA constructs.

FIGS. 6A-6D presents results for exon-skipping experiments in vivo in which U7_ACCA SC rAAV was delivered by intramuscular injection in Dup2 mice.

FIGS. 7A-7K are the rh74 genome sequence (SEQ ID NO: 1) wherein nucleotides 210-2147 are the Rep 78 gene open reading frame, 882-208 are the Rep52 open reading frame, 2079-2081 are the Rep78 stop, 2145-2147 are the Rep78 stop, 1797-1800 are a splice donor site, 2094-2097 are a splice acceptor site, 2121-2124 are a splice acceptor site, 174-181 are the p5 promoter+1 predicted, 145-151 are the p5 TATA box, 758-761 are the p19 promoter+1 predicted, 732-738 are the p19 TATA box, 1711-1716 are the p40 TATA box, 2098-4314 are the VP1 Cap gene open reading frame, 2509-2511 are the VP2 start, 2707-2709 are the VP3 start and 4328-4333 are a polyA signal.

FIG. 8 shows a map of a plasmid with an AAV genome insert of an exemplary exon 2-targeted U7snRNA.

FIG. 9 shows the DNA sequence of the AAV genome insert (SEQ ID NO: 2) of the plasmid of FIG. 8.

FIG. 10 shows vertical bars indicating the approximate position of an MLPA probe.

FIG. 11 shows a schematic of a vector used in creation of a mdx^{dup2} (Dup2) mouse.

FIGS. 12A-12E show the results of intramuscular delivery to Dup2 mice of AAV1 U7-ACCA.

FIGS. 13A-13F show the results of intravenous injection of AAV9 U7_ACCA in the Dup2 mouse model.

### EXAMPLES

Aspects and embodiments of the invention are illustrated by the following examples.

### Example 1

### Isolation of AAV rh.74

A unique AAV serotype was isolated from a rhesus macaque lymph node using a novel technique termed Linear Rolling Circle Amplification. Using the LRCA process, double-stranded circular AAV genomes were amplified from several rhesus macaques. The method is predicated on the ability to amplify circular AAV genomes by isothermic rolling circle amplification using phi29 phage DNA polymerase and AAV specific primers. LRCA products are contiguous head-to-tail arrays of the circular AAV genomes from which full-length AAV Rep-Cap molecular clones were isolated. Four isolates were sequenced and the predicted amino acid sequences for Rep and Cap ORFs were aligned and compared to previously published serotypes (Table). VP1 protein sequences were analyzed and revealed homology to the NHP AAV clades D, E, and AAV 4-like virus isolates. Analysis of the Rep78 (top portion of Table) ORF revealed strong homology to AAV 1 (98-99%).

TABLE 1

| | AAV 1 | AAV 4 | AAV 7 | AAV 8 | rh.73 | rh.74 | rh.75 | rh.76 |
|---|---|---|---|---|---|---|---|---|
| AAV 1 | | 90 | 98 | 95 | 98 | 98 | 99 | |
| AAV 4 | 63 | | 90 | 87 | 90 | 90 | 90 | |
| AAV 7 | 85 | 63 | | 96 | 97 | 98 | 98 | |
| AAV 8 | 84 | 63 | 88 | | 97 | 97 | 95 | |
| rh.73 | 79 | 61 | 83 | 80 | | 99 | 99 | |
| rh.74 | 84 | 63 | 88 | 93 | 80 | | 99 | |
| rh.75 | 65 | 82 | 82 | 64 | 62 | 64 | | |
| rh.76 | 85 | 63 | 91 | 86 | 84 | 86 | 84 | |

Similarity of published AAV sequences and the new AAV sequences determined using one-pair alignment according to the Lipman-Pearson method implemented in the MegAlgn software in DNASTAR (DNASTAR Inc.). Light faced numbers (top, right) represent similarity in Rep78 sequences, whereas bold-faced numbers (lower, left) represent similarity in VP1 capsid sequences.

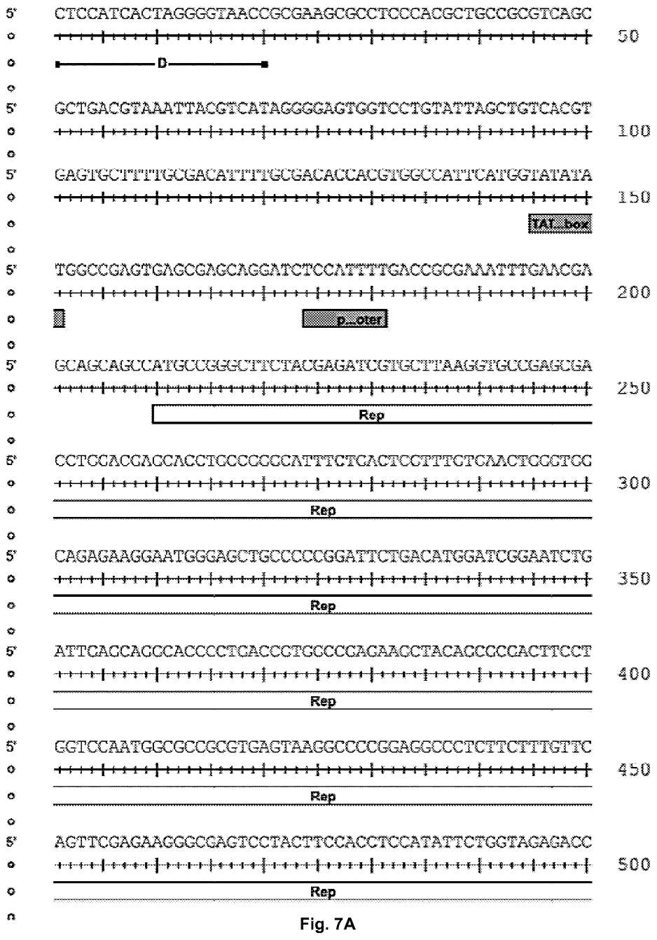

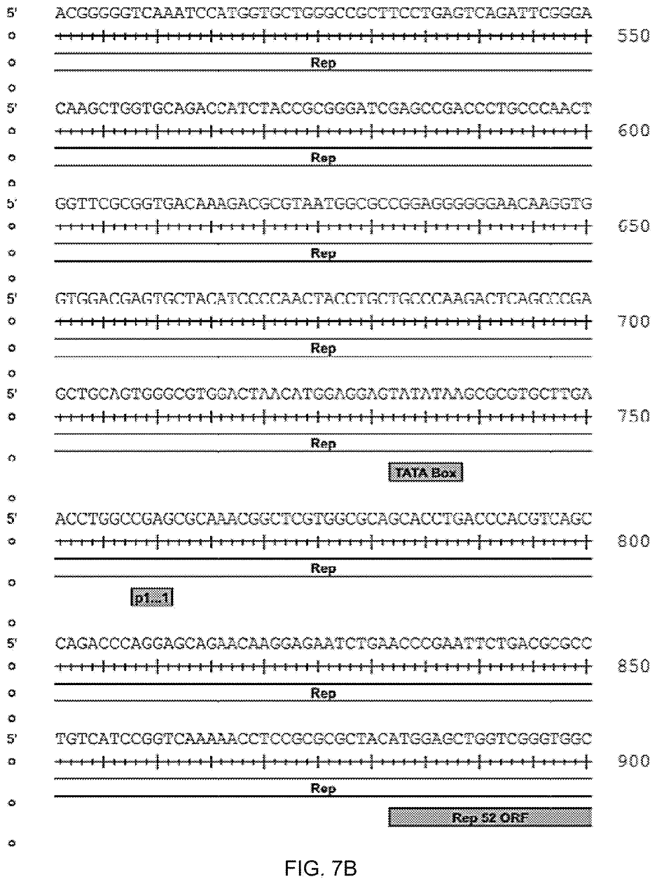

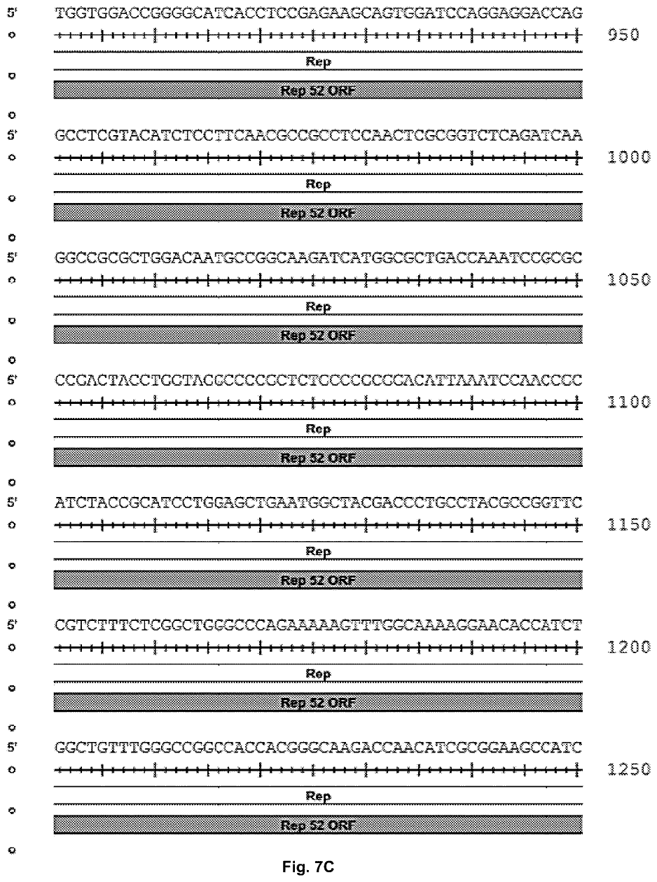

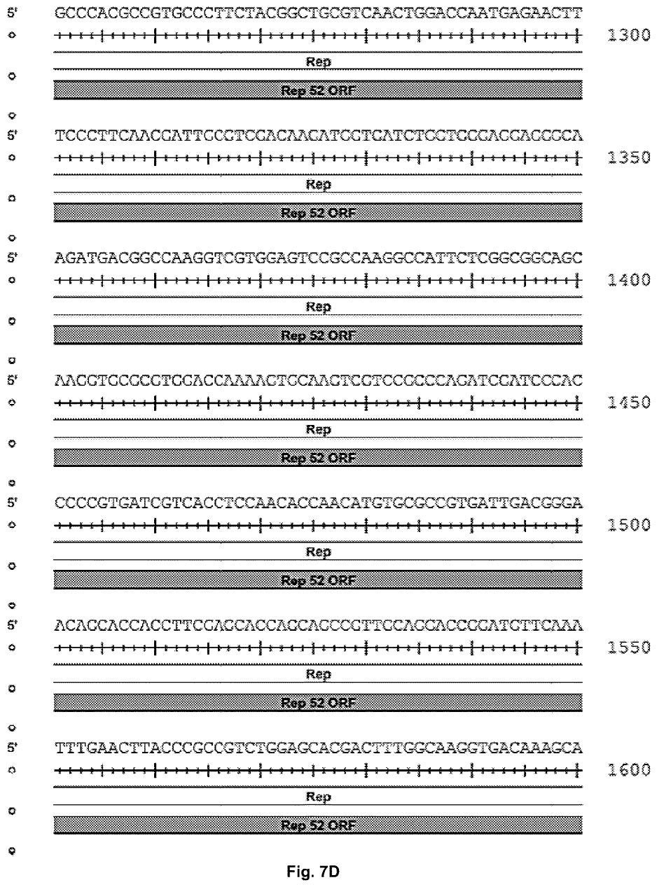

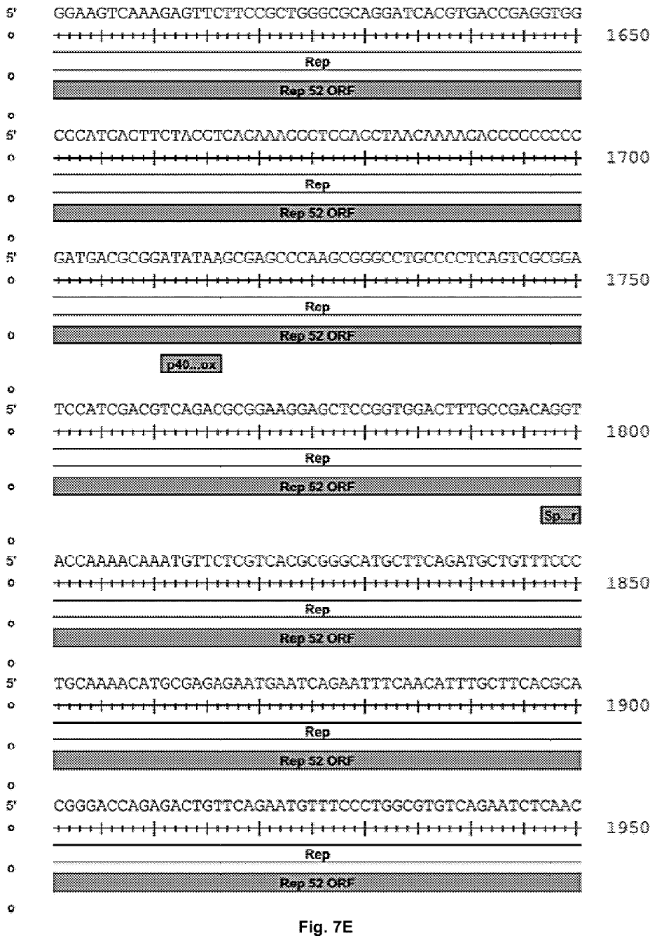

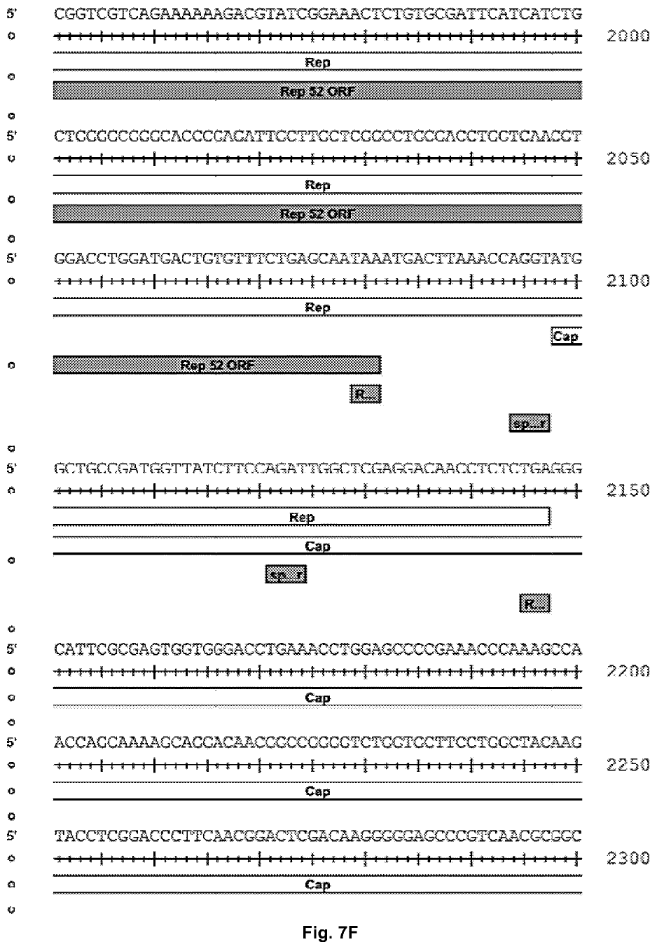

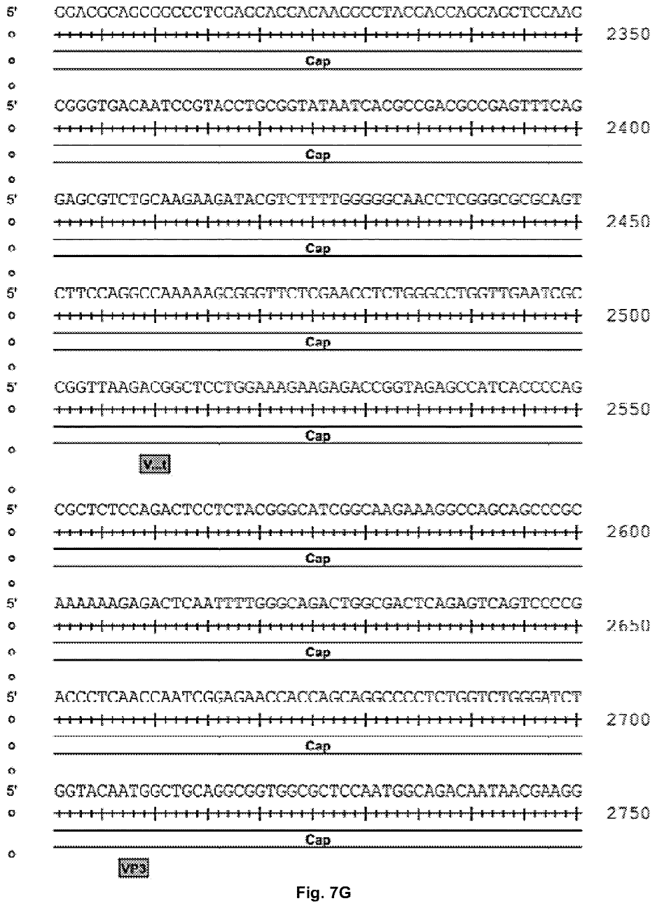

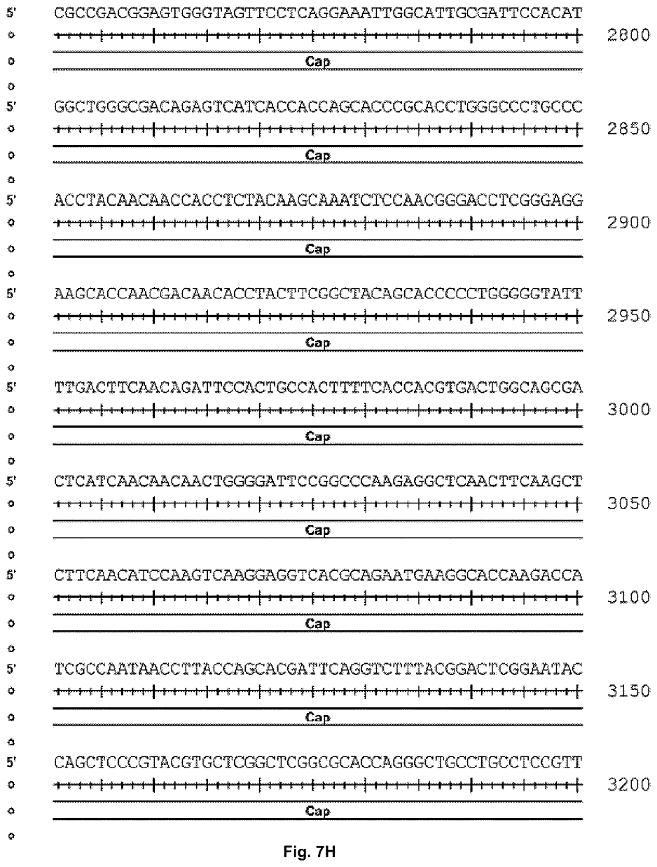

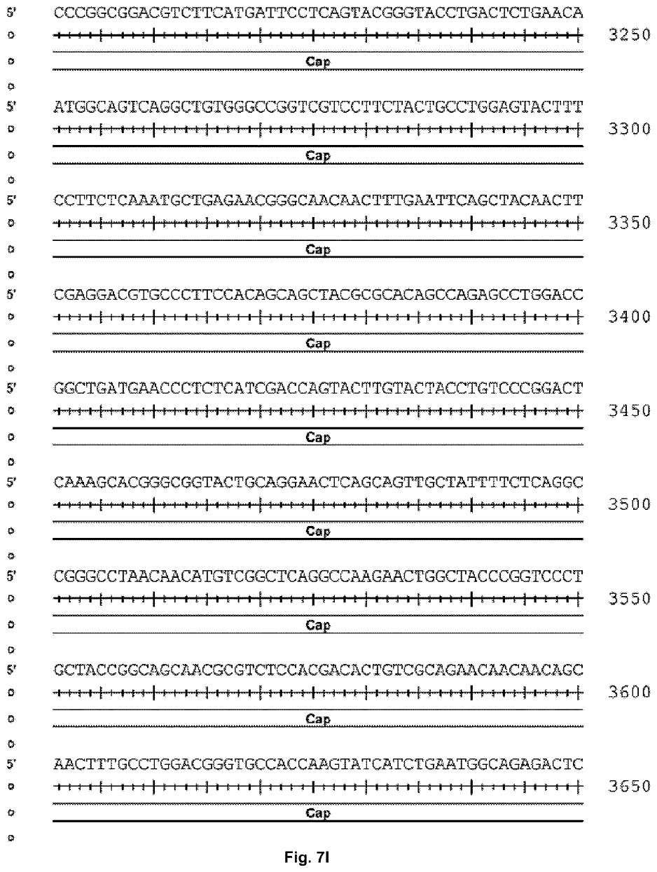

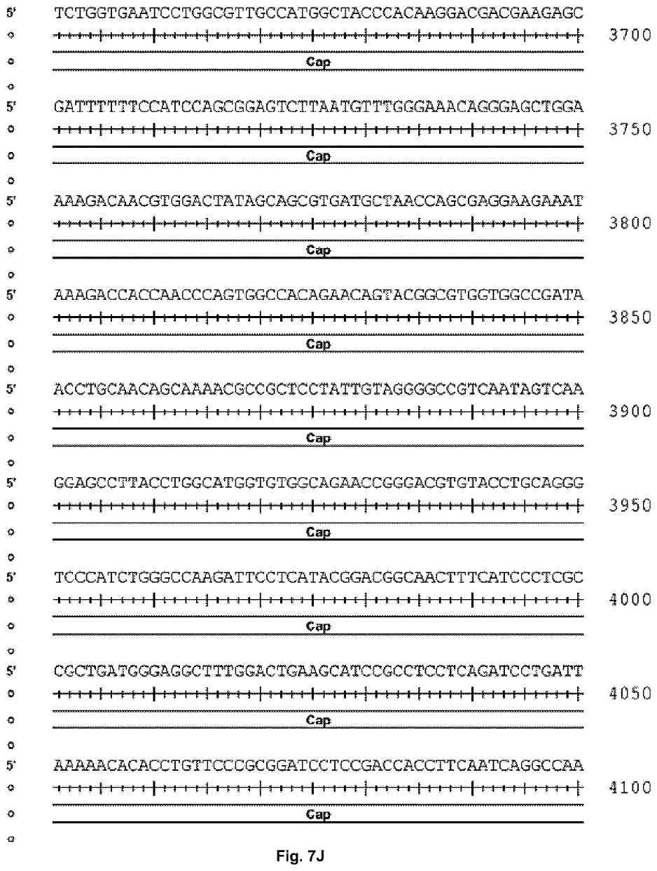

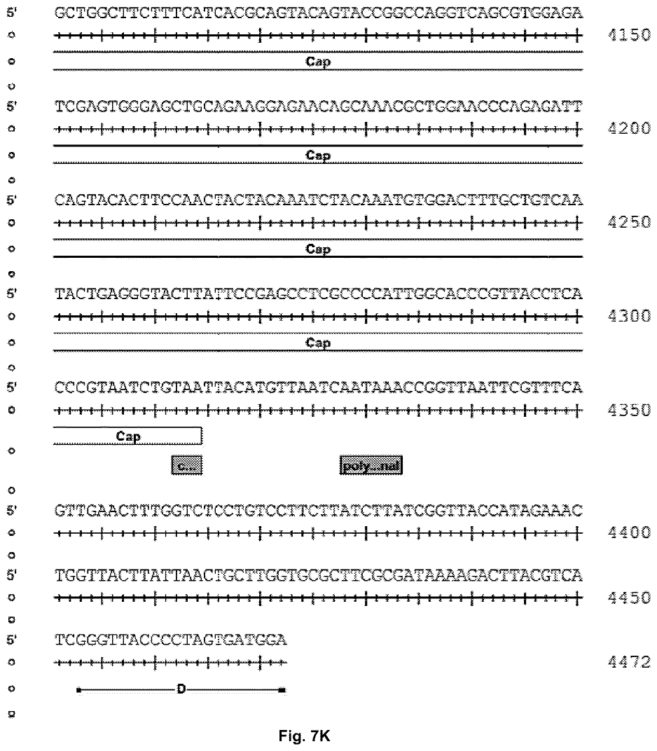

One macaque tissue sample (rh426-M) yielded a divergentAAV8-like isolate termed rh.74 that shares 93% sequence identity with AAV8. The nucleotide sequence of the rh.74 genome is set out in FIGS. 7A-7K and in SEQ ID NO: 1.

The rh.74 capsid gene sequence was cloned into an AAV helper plasmid containing the Rep gene from AAV2 to provide vector replication functions for recombinant AAV vector production.

### Example 2

### DMD Models

Examples of models of the DMD exon 2 duplication include in vivo and in vitro models as follows.

mdx^{dup2} Mouse Model

Mice carrying a duplication of exon 2 within the Dmd locus were developed. The exon 2 duplication mutation is the most common human duplication mutation and results in relatively severe DMD.

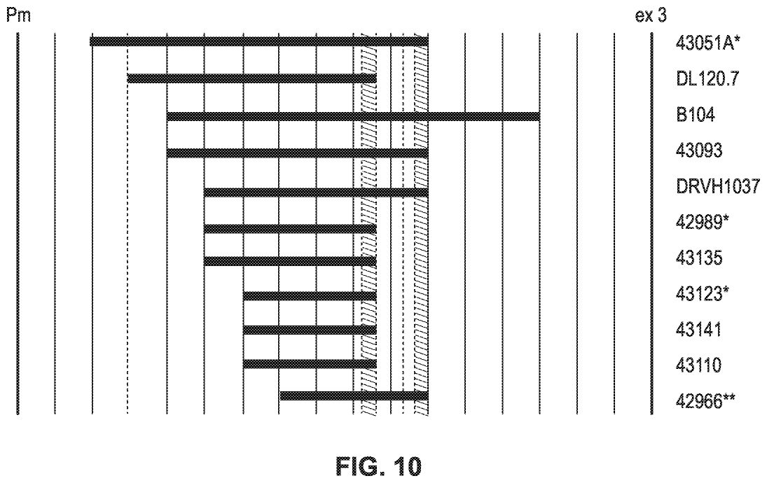

First, from White et al., Hum. Mutat, 27(9): 938-945 (2006), the maximum extent of the 11 different human exon 2 duplications was examined by MLPA and long-range PCR. Results are shown in FIG. 10. In FIG. 10, each vertical bar indicates the approximate position of an MLPA probe. The shaded columns indicate the two hotspot regions identified; they were used to determine the location of the insertion by homology of an exon 2 cassette in mouse.

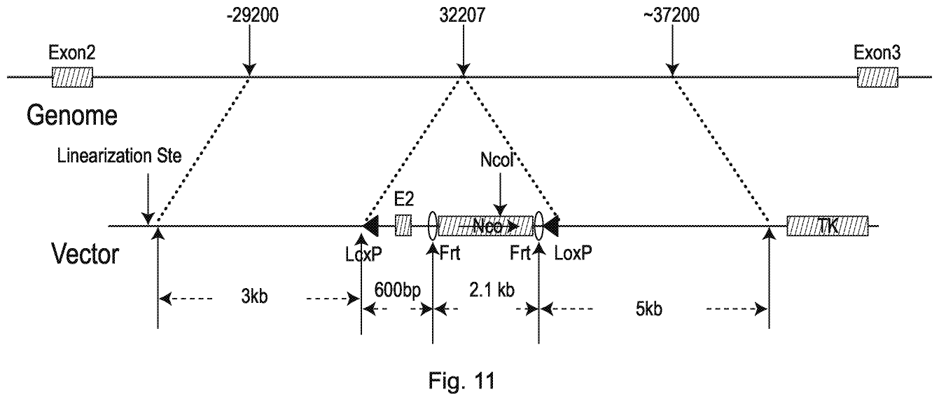

A map of the insertion vector is shown in FIG. 11. In the map, the numbers indicate the relative positions of cloning sites and exons and restriction sites. The neo cassette is in the same direction of the gene and the insertion point is precisely at 32207/32208 bp in the intron2. At least 150 bp extra intronic sequences are kept on each side of inserted exon 2, E2 region is 1775-2195 bp. Sizes of exon 2 and intron 2 are 62 bp and 209572 bp respectively.

Male C57BL/6 ES cells were transfected with the vector carrying the exon2 construct and then insertion was checked by PCR. One good clone was found, amplified and injected in dozens of albino BL/6 blastocysts. Injected blastocysts were implanted into recipient mice. The dystrophin gene from chimeric males was checked by PCR and then by RT-PCR. The colony was expanded and includes some female mice bred to homozygosity.

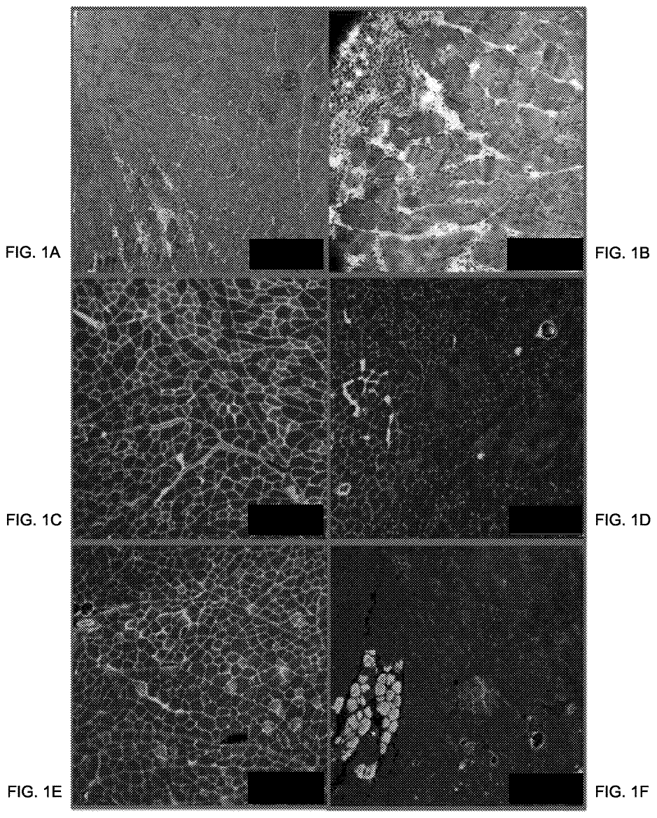

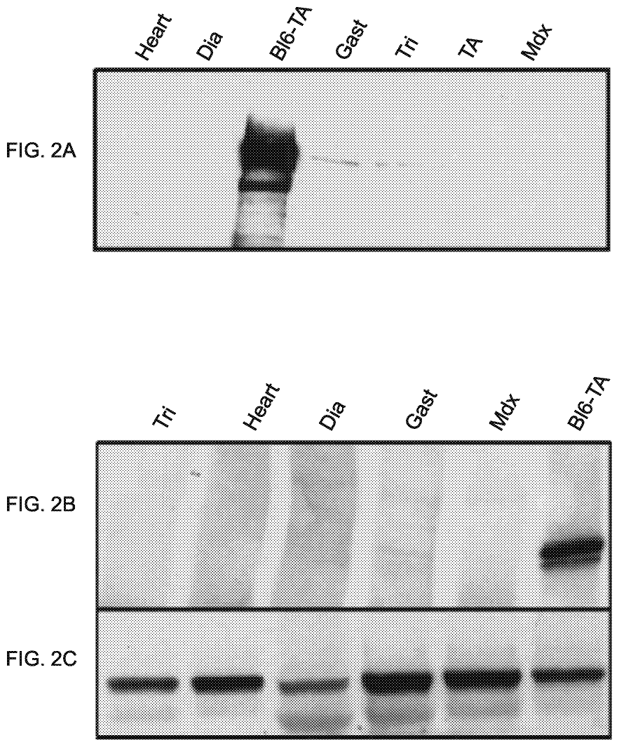

FIGS. 1A-1F and FIGS. 2A-2C demonstrate the dystrophin expression in muscles from a 4 week old hemizygous mdxdup2 mouse is essentially absent. (As seen in FIGS. 2A-2C, traces of expression can be detected using an C-terminal antibody but not the exon 1-specific Manex1A antibody, consistent with a very small amount of translation from the exon 6 alternate translational initiation site we previously described.)

Immortalized and Conditionally Inducible fibroMyoD Cell Lines

Expression of the MyoD gene in mammalian fibroblasts results in transdifferentiation of cells into the myogenic lineage. Such cells can be further differentiated into myotubes, and they express muscle genes, including the DMD gene.

Immortalized cell lines that conditionally express MyoD under the control of a tetracycline-inducible promoter were generated. This is achieved by stable transfection of the primary fibroblast lines of a lentivirus the tet-inducible MyoD and containing the human telomerase gene (TER). The resultant stable line allows MyoD expression to be initiated by treatment with doxycycline. Such cell lines were generated from patients with DMD who carry a duplication of exon 2.

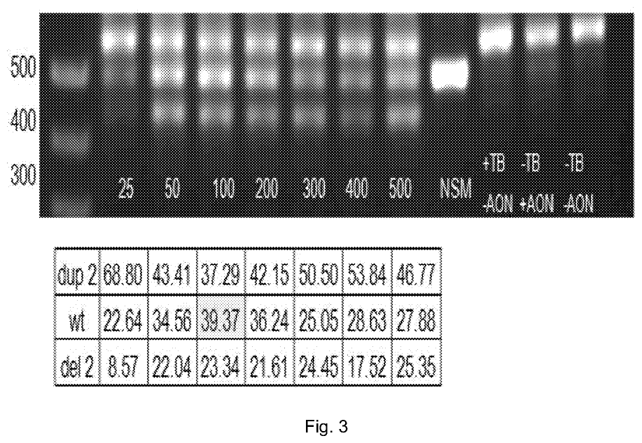

Using the line, duplication skipping using 2′-O-methyl antisense oligomers (AONs) provided by Dr. Steve Wilton (Perth, Australia) was demonstrated. Multiple cell lines were tested. Results from exemplary cells lines are shown in FIG. 3.

### Transiently MyoD-Transfected Primary Cell Lines

Proof-of-principle experiments using primary fibroblast lines transiently transfected with adenovirus-MyoD were conducted. The adenovirus constructs were not integrated in the cell genomes, yet MyoD was transiently expressed. The resulting DMD expression was sufficient to perform exon skipping experiments (although reproducibility favors the stably transfected lines.)

### Example 3

Effectiveness of U7 snRNA-Mediated Skipping on Exon 2 Duplication Mutations

Products and methods for virally-mediated exon skipping of duplicated exons were developed. The products and methods were modified compared to the U7snRNA systems described in Goyenvalle et al., Science, 306(5702): 1796-1799 (2004) or Goyenvalle et al., Mol. Ther., 20(6): 179601799 (2004).

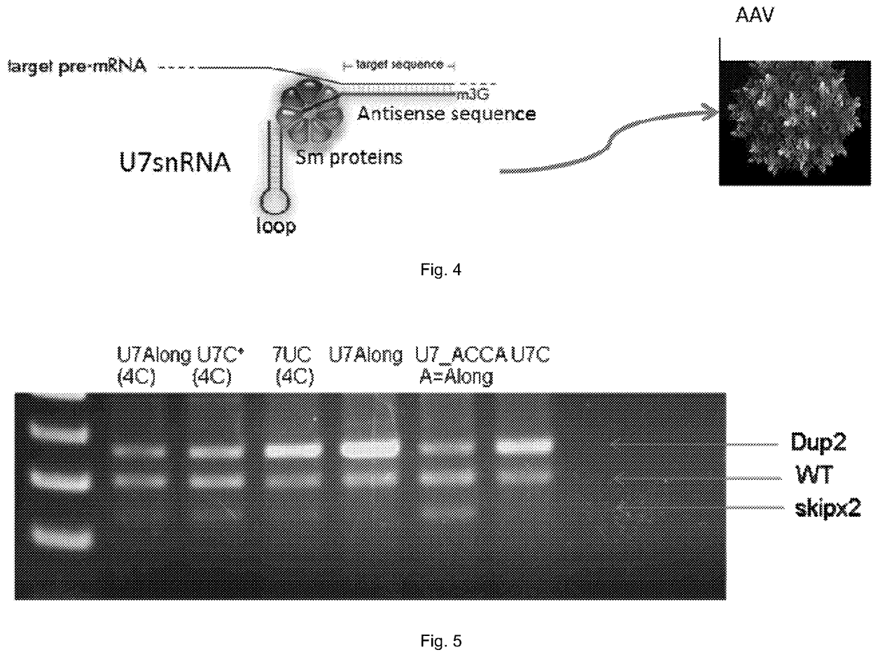

U7snRNA was modified to include a target antisense sequence to interfere with splicing at a given target exon (FIG. 4). Specifically, four new exon 2 targeting sequences were designed based upon the results of the AON studies described in Example 2.

U7B
(SEQ ID NO: 3)
TCAAAAGAAAACATTCACAAAATGGGTA
U7Along
(SEQ ID NO: 4)
GTTTTCTTTTGAAGATCTTCTCTTTCATcta
U7Ashort
(SEQ ID NO: 5)
AGATCTTCTCTTTCATcta
U7C
(SEQ ID NO: 6)
GCACAATTTTCTAAGGTAAGAAT

U7 snRNA constructs including the exon 2 target sequences were generated. Each U7 snRNA construct included one of the target sequences. U7 snRNA constructs targeted to selected other exons were also generated (based upon MyoD-transdifferentiated cell line studies, above). Self complementary (SC) AAV vectors with genomes including one or more of the U7 snRNA constructs were then produced.

For experiments in cell culture and for intramuscular injection in Dup2 mice, rAAV1 vectors were utilized. Recombinant SC AAV vectors of a desired AAV serotype were produced by a modified cross-packaging approach using a plasmid comprising a desired vector genome by an adenovirus-free, triple plasmid DNA transfection (CaPO₄ precipitation) method in HEK293 cells [Rabinowitz et al., J. Virol., 76:791-801 (2002)]. Vector was produced by co-transfecting with an AAV helper plasmid and an adenovirus helper plasmid in similar fashion as that previously described [Wang et al., Gene. Ther., 10:1528-1534 (2003)]. The adenovirus helper plasmid (pAdhelper) expresses the adenovirus type 5 E2A, E40RF6, and VA I/II RNA genes which are required for high-titer rAAV production.

Vectors were purified from clarified 293 cell lysates by sequential iodixanol gradient purification and anion-exchange column chromatography using a linear NaCl salt gradient as previously described [Clark et al., Hum. Gene Ther, 10:1031-1039 (1999)]. Vector genome (vg) titers were measured using QPCR based detection with a specific primer/probe set utilizing the Prism 7500 Taqman detector system (PE Applied Biosystems) as previously described (Clark et al., supra). Vector stock titers ranged between 1-10×10¹² vg/mL.

Initial exon-skipping analysis was by RT-PCR using the SC rAAV vectors to transduce Dup2 immortalized human fibromyoblasts. Dup 2 immortalized human fibroblasts that were able to transdifferentiate into muscle lineage cells under the control of doxycycline were produced by transduction with both telomerase-expressing and tet-inducible-MyoD expressing vectors. The converted human fibromyoblasts (FM) were then transduced with the SC rAAV carrying different U7 constructs incorporating exon 2 antisense sequences.

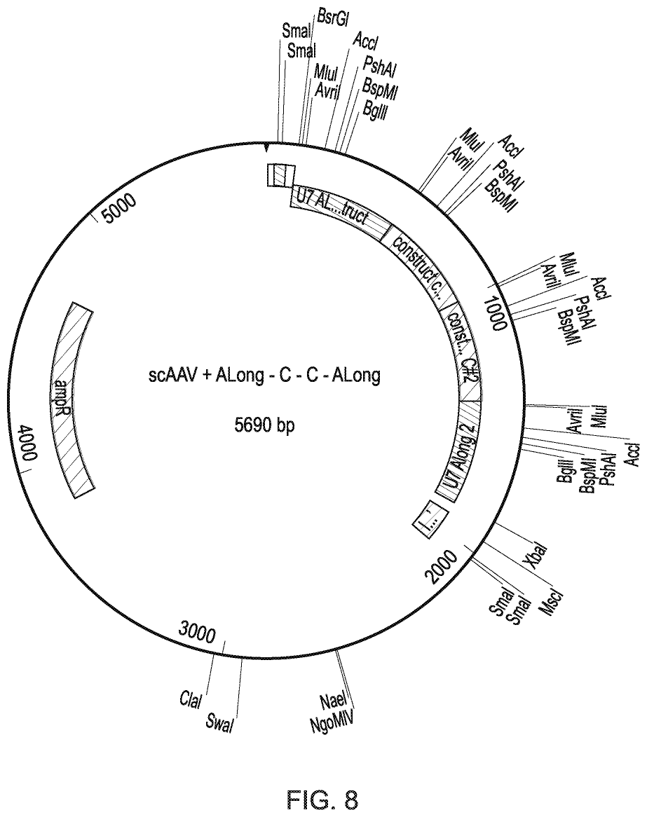

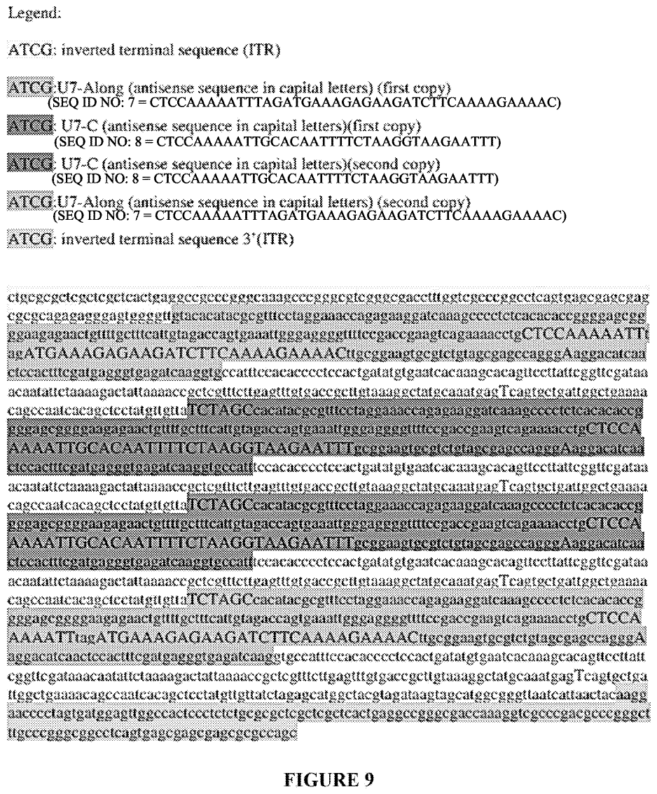

RT-PCR results are shown in FIG. 5 for SC rAAV.1-U7 constructs with three different antisense sequences. In FIG. 5, “(4C)” indicates four copies of the U7 construct were included in a vector genome, “+” indicates a higher dose and “U7_ACCA A=Along” indicates a vector genome (shown in a plasmid map in FIG. 8 and the sequence of which, SEQ ID NO: 2, is set out in FIG. 9) comprising in sequence four exon 2-targeted U7 snRNA polynucleotide constructs: a first U7Along construct, a first U7C construct, a second U7C construct and a second U7Along construct. As shown, the U7_ACCA A-Along SC rAAV (abbreviated U7_ACCA SC rAAV1 elsewhere herein) achieved a higher percentage of exon 2 skipping in comparison to any other vector construct.

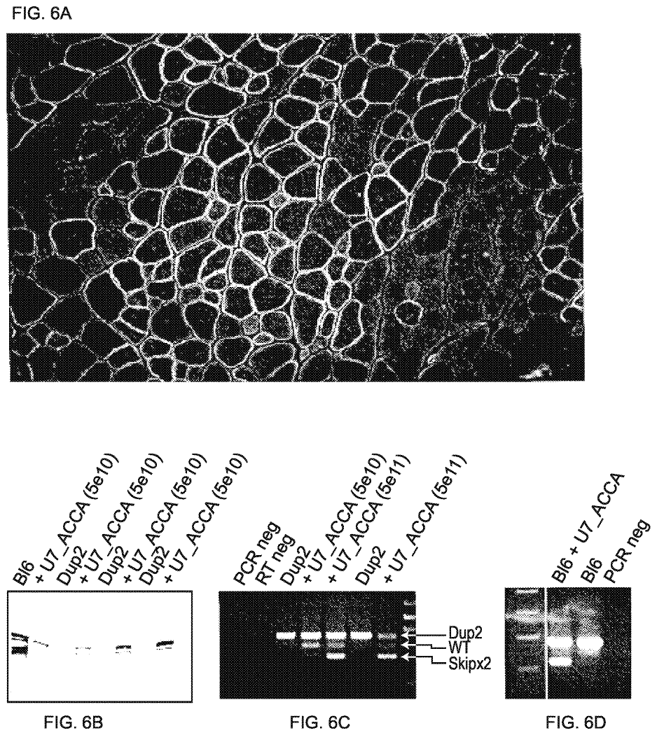

In subsequent experiments, exon-skipping efficiency was analyzed in vivo. The most efficient AAV-U7 vector, U7_ACCA SC rAAV1, was chosen for intramuscular injection in Dup2 mice. Results are shown below in FIGS. 6A-6D wherein (FIG. 6A) shows dystrophin staining where the protein expression is restored, and is properly localized at the membrane in many muscle fibers; (FIG. 6B) protein restoration was confirmed by western blot. RT-PCR shows (FIG. 6C) dose-dependent single or double skipping in Dup2 mice, as well as (FIG. 6D) efficient skipping in the wild-type mouse.

Thus, a highly efficient AAV-mediated U7snRNA was designed to skip exon 2 allowing subsarcolemmal dystrophin restoration. Cardiac function; EDL and diaphragm force assessments; and treadmill and grip tests will be compared between untreated and treated mice. Based upon the degree of dystrophin expression detectable within the injected muscle, U7_ACCA SC rAAV was chosen for further experiments to be delivered intravenously to a first cohort at 1E11 vg/kg, followed by dosing one log higher in a second cohort Injection will be performed at four weeks, and animals evaluated by physiologic assessment and histopathology at 10 and 24 weeks (n=8 animals per cohort) as described above.

### Example 4

### Intramuscular Delivery of U7-ACCA by AAV1 Results in Significant N-Truncated Dystrophin Expression in Dup2 Mice

A rAAV1 comprising the genome insert of FIG. 9 was produced by the methods described in Example 3. The AAV.1U7-ACCA was then administered to Dup2 mice via intramuscular injection.

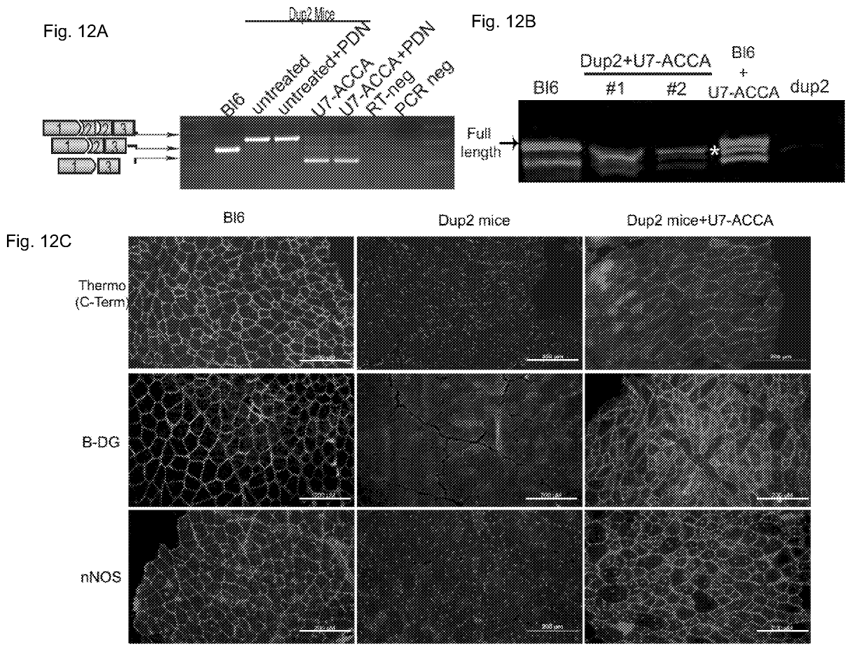

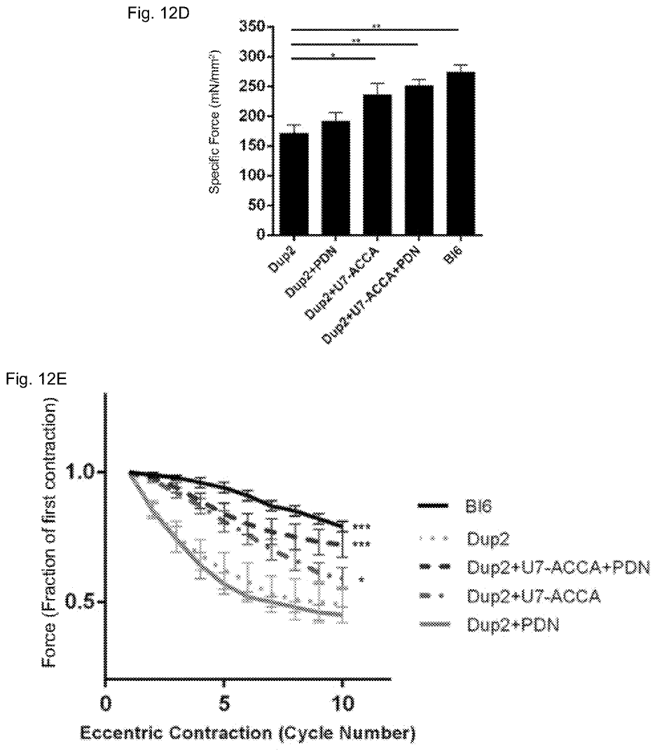

RT-PCR performed on DMD mRNA 4 weeks after TA intramuscular injection of 5e11vg AAV.1U7-ACCA showed nearly complete skipping of both copies of exon 2 in Dup2 animals [FIG. 12A].

Immunoblot using a C-terminal antibody (PA1-21011, ThermoScientific) performed a month after infection showed significant expression of the N-truncated isoform (asterisk) in both Dup2 and control Bl6 mice [FIG. 12B]. The protein induced in Bl6 males injected with U7-ACCA was of the same size as that expressed in the Dup2 treated animals, confirming the size difference between this protein and the full-length isoform.

Immunofluorescent staining of dystrophin, β-dystroglycan, and neuronal nitric oxide synthase demonstrated restoration of members of the dystrophin associated complex [FIG. 12C].

Normalized specific force following tetanic contraction in untreated Dup2 animals was significantly less than in Bl6 mice Intramuscular injection of AAV1.U7-ACCA, either alone or with prednisone, significantly increased force to levels that were not significantly different from that seen in Bl6 mice. No significant difference was observed between untreated Dup2 mice and those treated with prednisone along (Dup2+PDN) [FIG. 12D]. For this assay, normalized specific force was evaluated using a published protocol [Hakim et al., Journal of Applied Physiology, 110: 1656-1663 (2011)].

Treatment significantly protected Dup2 muscle from loss of force following repetitive eccentric contractions, as assessed by published protocols (Hakim et al., supra). Treatment of Dup2 mice with AAV1.U7-ACCA alone resulted in a statistically significant improvement compared to untreated Dup2 mice. The combination of AAV1.U7-ACCA and prednisone resulted in no significant difference in comparison to control Bl6 mice in force retention following contractions #3 to #10 [FIG. 12E].

### Example 5

### Intravenous Injection of AAV9-U7_ACCA in the Dup2 Mouse Model Results in Significant Expression of the N-Truncated Isoform and Correction of Strength Deficit

Based upon the degree of dystrophin expression detectable within injected muscle, we chose to deliver U7_ACCA SC rAAV intravenously for further experiments, and selected the serotype rAAV9 based upon known tissue distribution properties.

A rAAV9 comprising the genome insert of FIG. 9 was produced by the methods described in Example 3. The AAV.9U7-ACCA was then administered to Dup2 mice. A first cohort was injected via tail vein with 3.3E112 vg/kg. Injection was performed at four weeks of age.

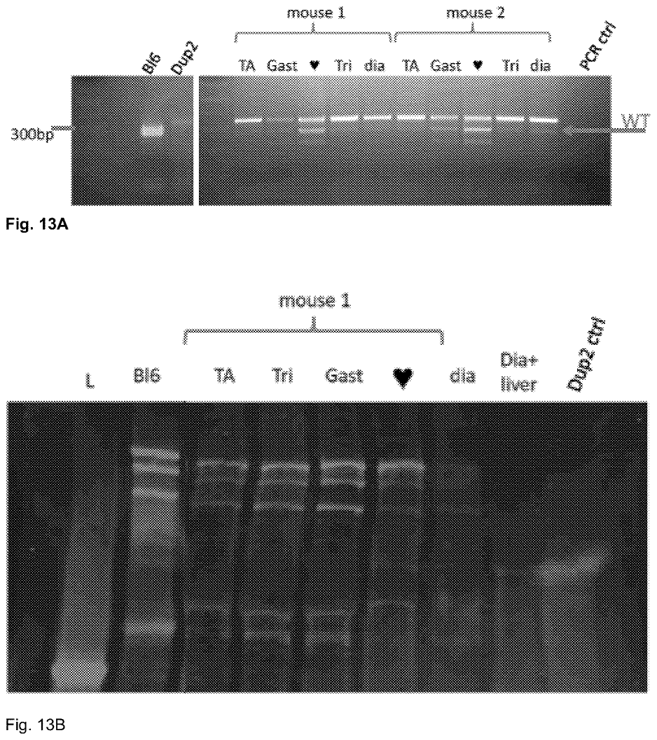

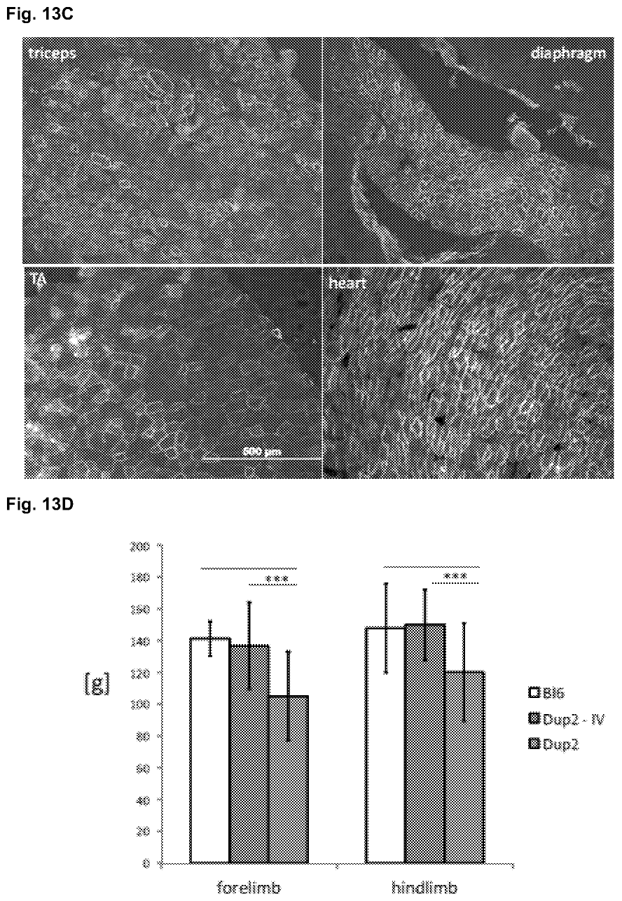

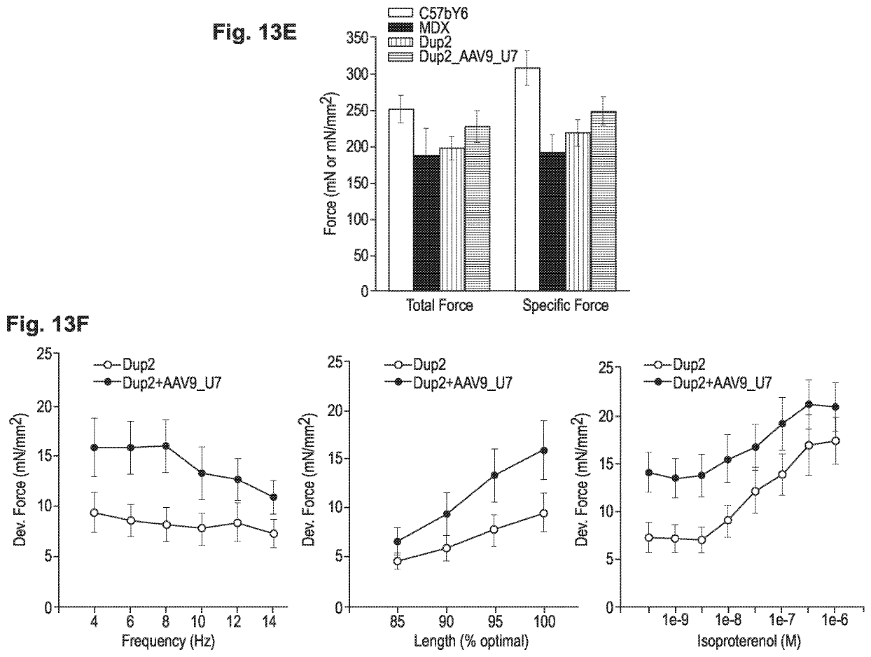

RT-PCR was performed on five different Dup2 mouse muscles one month after tail vein injection of AAV9.U7-ACCA (3.3E12 vg/kg) [FIG. 13A]. As demonstrated by the presence of multiple transcripts (labeled Dup2, wt, and Del2), U7-ACCA treatment was able to force skipping of one or both copies of exon 2 in all muscles tested. (TA: tibialis anterior; Gas: gastrocnemius; r: heart; Tri: triceps; dia: diaphragm.)

Western blot using a C-terminal antibody (PA1-21011, ThermoScientific) performed on five different muscles one month after injection demonstrated the presence of dystrophin in all tested muscles[FIG. 13B].

Immunostaining using a C-terminal antibody (PA1-21011, ThermoScientific) of dystrophin on the same samples confirmed dystrophin expression and its proper localization at the sarcolemma [FIG. 13C].

Evaluation of both forelimb and hindlimb grip strength demonstrated a complete correction of grip strength in Dup2 animals treated with AAV9.U7-ACCA [FIG. 13D]. This assay was performed using a published protocol [Spurney, et al., Muscle & Nerve, 39, 591-602 (2009)].

Normalized specific and total forces following tetanic contraction showed improvement in muscle force in comparison to untreated Dup2 animals [FIG. 13E], using a published protocol [Hakim et al., supra).

Cardiac papillary muscles demonstrated improvements in length-dependent force generation in treated animals [FIG. 13F], using a published protocol [Janssen et al., Am J Physiol Heart Circ Physiol., 289(6):H2373-2378 (2005)].

While the present invention has been described in terms of specific embodiments, it is understood that variations and modifications will occur to those skilled in the art. Accordingly, only such limitations as appear in the claims should be placed on the invention.

All documents referred to in this application are hereby incorporated by reference in their entirety with particular attention to the content for which they are referred.

We claim:

1. A nucleic acid comprising at least one nucleotide sequence or a combination of two or more exon 2-targeted nucleotide sequences, wherein each nucleotide sequence is set forth in any one of SEQ ID NOs: 4, 5, 7 and 8.

2. The nucleic acid of claim 1 comprising the nucleotide sequence set forth in SEQ ID NO: 4.

3. The nucleic acid of claim 1 comprising the nucleotide sequence set forth in SEQ ID NO: 5.

4. The nucleic acid of claim 1 comprising the nucleotide sequence set forth in SEQ ID NO: 7.

5. The nucleic acid of claim 1 comprising the nucleotide sequence set forth in SEQ ID NO: 8.

6. The nucleic acid of claim 1, wherein the combination comprises two or more nucleotide sequences, each as set forth in SEQ ID NO: 4.

7. The nucleic acid of claim 1, wherein the combination comprises two or more nucleotide sequences, each as set forth in SEQ ID NO: 5.

8. The nucleic acid of claim 1, wherein the combination comprises two or more nucleotide sequences, each as set forth in SEQ ID NO: 7.

9. The nucleic acid of claim 1, wherein the combination comprises two or more nucleotide sequences, each as set forth in SEQ ID NO: 8.

10. The nucleic acid of claim 1, wherein the combination comprises the nucleotide sequence set forth in SEQ ID NO: 7 and the nucleotide sequence set forth in SEQ ID NO: 8.

11. The nucleic acid of claim 1, wherein the combination comprises in sequence four exon 2-targeted nucleotide sequences comprising a first U7Along antisense construct comprising the nucleotide sequence set out in SEQ ID NO: 7, a first U7C antisense construct comprising the nucleotide sequence set out in SEQ ID NO: 8, a second U7C antisense construct comprising the nucleotide sequence set out in SEQ ID NO: 8, and a second U7Along antisense construct comprising the nucleotide sequence set out in SEQ ID NO: 7.

12. A nucleic acid comprising a U7snRNA polynucleotide construct comprising the nucleic acid of claim 1.

13. A composition comprising the nucleic acid of claim 1 and a carrier or diluent.

14. A method of ameliorating Duchenne Muscular Dystrophy in a patient with DMD exon 2 duplications in need thereof comprising administering the nucleic acid of claim 1 to the patient.

15. The method of claim 14, wherein the ameliorating results in increased expression of dystrophin protein in the patient.

16. The method of claim 14, wherein the ameliorating inhibits the progression of dystrophic pathology in the patient.

17. The method of claim 14, wherein the ameliorating improves muscle function in the patient.

18. The method of claim 14, wherein the improvement in muscle function is an improvement in muscle strength.

19. The method of claim 14, wherein the improvement in muscle function is an improvement in stability in standing and walking.


SEQUENCE LISTING (8 sequences)

SEQ ID NO: 1 - DNA, 4472 bp, Adeno-associated virus
  source 1..4472: mol_type = genomic DNA
ctccatcactaggggtaaccgcgaagcgcctcccacgctgccgcgtcagcgctgacgtaaattacgtcataggggagtggtcctgtattagctgtcacgtgagtgcttttgcgacattttgcgacaccacgtggccattcatggtatatatggccgagtgagcgagcaggatctccattttgaccgcgaaatttgaacgagcagcagccatgccgggcttctacgagatcgtgcttaaggtgccgagcgacctggacgagcacctgccgggcatttctgactcgtttgtgaactgggtggcagagaaggaatgggagctgcccccggattctgacatggatcggaatctgattgagcaggcacccctgaccgtggccgagaagctacagcgcgacttcctggtccaatggcgccgcgtgagtaaggccccggaggccctcttctttgttcagttcgagaagggcgagtcctacttccacctccatattctggtagagaccacgggggtcaaatccatggtgctgggccgcttcctgagtcagattcgggacaagctggtgcagaccatctaccgcgggatcgagccgaccctgcccaactggttcgcggtgacaaagacgcgtaatggcgccggaggggggaacaaggtggtggacgagtgctacatccccaactacctgctgcccaagactcagcccgagctgcagtgggcgtggactaacatggaggagtatataagcgcgtgcttgaacctggccgagcgcaaacggctcgtggcgcagcacctgacccacgtcagccagacccaggagcagaacaaggagaatctgaacccgaattctgacgcgcctgtcatccggtcaaaaacctccgcgcgctacatggagctggtcgggtggctggtggaccggggcatcacctccgagaagcagtggatccaggaggaccaggcctcgtacatctccttcaacgccgcctccaactcgcggtctcagatcaaggccgcgctggacaatgccggcaagatcatggcgctgaccaaatccgcgcccgactacctggtaggccccgctctgcccgcggacattaaatccaaccgcatctaccgcatcctggagctgaatggctacgaccctgcctacgccggttccgtctttctcggctgggcccagaaaaagtttggcaaaaggaacaccatctggctgtttgggccggccaccacgggcaagaccaacatcgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgatcccacccccgtgatcgtcacctccaacaccaacatgtgcgccgtgattgacgggaacagcaccaccttcgagcaccagcagccgttgcaggaccggatgttcaaatttgaacttacccgccgtctggagcacgactttggcaaggtgacaaagcaggaagtcaaagagttcttccgctgggcgcaggatcacgtgaccgaggtggcgcatgagttctacgtcagaaagggtggagctaacaaaagacccgcccccgatgacgcggatataagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacatgcgagagaatgaatcagaatttcaacatttgcttcacgcacgggaccagagactgttcagaatgtttccctggcgtgtcagaatctcaaccggtcgtcagaaaaaagacgtatcggaaactctgtgcgattcatcatctgctggggcgggcacccgagattgcttgctcggcctgcgacctggtcaacgtggacctggatgactgtgtttctgagcaataaatgacttaaaccaggtatggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcactaaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaattacatgttaatcaataaaccggttaattcgtttcagttgaactttggtctcctgtccttcttatcttatcggttaccatagaaactggttacttattaactgcttggtgcgcttcgcgataaaagacttacgtcatcgggttacccctagtgatgga

SEQ ID NO: 2 - DNA, 2052 bp, Adeno-associated virus
  misc_feature 1..88: note = Inverted Terminal Sequence (ITR)
  misc_feature 89..365: note = U7-Along
  misc_feature 525..806: note = U7-C
  misc_feature 961..1242: note = U7-C
  misc_feature 1397..1678: note = U7-Along
  misc_feature 1830..2052: note = Inverted Terminal Sequence 3' (ITR)
  source 1..2052: mol_type = genomic DNA
ctgcgcgctcgctcgctcactgaggccgcccgggcaaagcccgggcgtcgggcgacctttggtcgcccggcctcagtgagcgagcgagcgcgcagagagggagtggggttgtacacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaatttagatgaaagagaagatcttcaaaagaaaacttgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgttatctagccacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaattgcacaattttctaaggtaagaatttgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgttatctagccacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaattgcacaattttctaaggtaagaatttgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgttatctagccacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaatttagatgaaagagaagatcttcaaaagaaaacttgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgttatctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagc

SEQ ID NO: 3 - DNA, 28 bp, Adeno-associated virus
  source 1..28: mol_type = genomic DNA
tcaaaagaaaacattcacaaaatgggta

SEQ ID NO: 4 - DNA, 31 bp, Adeno-associated virus
  source 1..31: mol_type = genomic DNA
gttttcttttgaagatcttctctttcatcta

SEQ ID NO: 5 - DNA, 19 bp, Adeno-associated virus
  source 1..19: mol_type = genomic DNA
agatcttctctttcatcta

SEQ ID NO: 6 - DNA, 23 bp, Adeno-associated virus
  source 1..23: mol_type = genomic DNA
gcacaattttctaaggtaagaat

SEQ ID NO: 7 - DNA, 42 bp, Adeno-associated virus
  source 1..42: mol_type = genomic DNA
ctccaaaaatttagatgaaagagaagatcttcaaaagaaaac

SEQ ID NO: 8 - DNA, 36 bp, Adeno-associated virus
  source 1..36: mol_type = genomic DNA
ctccaaaaattgcacaattttctaaggtaagaattt